(19) United States
(12) Patent Application Publication
(10) Pub. No.: US 2007/0232979 A1
(43) Pub. Date: Oct. 4, 2007

(54) Decorative bandages and covers

(76) Inventor: Janet H. Montgomery, Naples, FL (US)

Correspondence Address: EDWARD M. LIVINGSTON, PA, 963 TRAIL TERRACE DRIVE, NAPLES, FL, 34103, US

(21) Appl. No.: 11/391,074

(22) Filed: Mar. 28, 2006

(51) Int. Cl. A61F 15/00; A61F 13/00

(52) U.S. Cl. 602/58; 602/41

(57) ABSTRACT

A decorative bandage (1) or cover (6) having a top surface (2) decorated with decorative objects, such as crystals (4), glitter (8), beads and the like, and a self-adhesive bottom surface (3). To use, a person simply applies the decorative bandage (1) or cover (6) on the object to be hidden. The object to be hidden may be an injury or blemish on the person, such as a cut, scrape or zit, or a blemish on an object, such as a stain on clothing. Because the top surface (2) is decorated with decorative objects, the bandage (1) or cover (6) gives the appearance of raised artwork that is applied directly onto a person or object, rather than a flat bandage or cover.

### BACKGROUND OF THE INVENTION

This invention relates to bandages and covers, more particularly, bandages and covers that are attractive to wear and are not indicative of the fact that a person is masking an injury, blemish or a spill or spot on clothing.

Self-adhesive bandages have been used in the public for many, many years. Although they suit the purpose of covering wounds and providing a cushion from further injury of a wound or blemish, the bandages themselves are often unattractive. Although the bandages are usually tan-colored so as to “blend” in with the skin color of a wearer, the shade of the bandage is usually very different from the shade of the wearer's skin, thereby defeating the purpose of “blending-in.” Inevitably, the injured person is often asked the question “what happened?”.

Over the recent years, however, bandages have become more aesthetically pleasing to the public as a whole. Clear bandages have become very popular as they permit a person's skin coloring to show through the adhesive portion of the bandage, thereby drastically reducing the appearance of the bandage. However, although the adhesive portion of the bandage is clear, light still reflects off of the clear portion, thereby inviting individuals to notice the bandage and still ask “what happened.”

In addition, bandages which are aesthetically pleasing to individuals who like to show off the bandages, such as children, have also become very popular. Bandages having bright colors, patterns and even cartoon characters are sought after by the individuals who like being asked “what happened?”.

Although the recent progression of decorative bandages have allowed individuals to either de-emphasize or exaggerate the fact that they are wearing a bandage, the fact remains that others know that a person is wearing a bandage and ask “what happened?”. Although children may enjoy this type of attention, oftentimes adults do not.

In addition, many individuals accidently spill foods and/or beverages on his or her clothing while dining. Although a person may try to wash off the stain with soap and water or with a liquid spot remover treatment, the process for removing a stain almost always leaves a wet mark on the clothing, thereby making the spot or stain more noticeable. If the spill occurs in private, then it is easy to take care of the stain and change clothes. However, if out in public, individuals are often faced with the difficult decision of either leaving the spot or stain as is or attempt to remove the stain and create a large wet mark on the clothing.

Thus, a need exists for a decorative bandage and cover that permits a person to mask an injury, blemish, spill or spot without permitting others to realize that the wearer has such.

The relevant prior art includes the following references:

| Patent No. | | |
|---|---|---|
| (U.S. unless stated otherwise) | Inventor | Issue/Publication Date |
| 4,285,338 | Lemelson | Aug. 25, 1981 |
| 6,586,651 | Sullivan | Jul. 01, 2003 |
| 2002/0069671 | Richardson | Jun. 13, 2002 |
| Des. 368,526 | Palermo | Apr. 02, 1996 |
| Des. 392,045 | Palermo | Mar. 10, 1998 |
| Des. 419,678 | Gadison | Jan. 25, 2000 |
| 4,212,296 | Schaar | Jul. 15, 1980 |
| D476,412 | Berry | Jun. 24, 2003 |

### SUMMARY OF THE INVENTION

The primary object of the present invention is to provide a decorative bandage and cover that does not permit other individuals to realize that a person is wearing a bandage or cover.

A further object of the present invention is to provide a decorative bandage and cover that is attractive to wear.

An even further object of the present invention is to provide a decorative bandage and cover that masks an injury or blemish.

An additional object of the present invention is to provide a decorative cover to hide spills and spots on clothing.

The present invention fulfills the above and other objects by providing a decorative bandage and cover having a top surface and a self-adhesive bottom surface. The top surface is decorated with a decorative object, such as crystals, glitter, beads and the like, on the so as to give the appearance of raised artwork when the bandage or cover is applied directly onto a person or object. To use, a person simply applies the decorative bandage and cover on the object to be hidden. The object to be hidden may be an injury or blemish on the person, such as a cut, scrape or zit or blemish or a spill or spot on an object, such as a stain on clothing.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a front view of a decorative bandage of the present invention;

FIG. 2 is a side view of the embodiment of FIG. 1;

FIG. 3 is a front view of a decorative cover of the present invention;

FIG. 4 is a side view of the embodiment of FIG. 2; and

FIG. 5 is a perspective view of a person wearing both the decorative bandage and decorative cover of the present invention.

### DESCRIPTION OF THE PREFERRED EMBODIMENTS

For purposes of describing the preferred embodiment, the terminology used in reference to the numbered components in the drawings is as follows:
1. decorative bandage
2. top surface
3. bottom surface
4. crystals
5. cushion
6. decorative cover
7. symbol
8. glitter

With reference to FIG. 1, a side view of a decorative bandage 1 of the present invention is shown. The bandage 1 includes a plurality of decorative objects, such as crystals 4 located on a top surface 2 of the decorative bandage 1. Although crystals 4 are shown, the decorative objects may also include gems, beads, pearls or any other decorative item. In addition, the decorative objects may be planar, two-dimensional or three-dimensional.

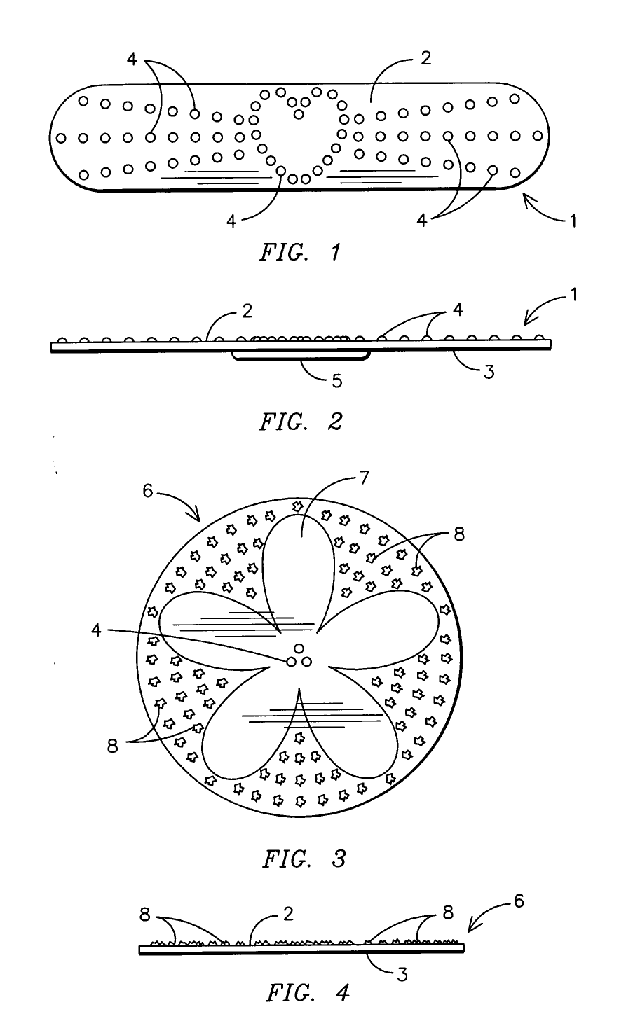

With reference to FIG. 2, a side view of a decorative bandage 1 of the present invention is shown. The decorative bandage 1 has a top surface 2 and a bottom surface 3. The bottom surface 3 is adhesive so as to adhere to a surface, such as a person's skin. A cushion 5 is located on the bottom surface 3 so as to protect an injury or would that the decorative bandage 1 is covering.

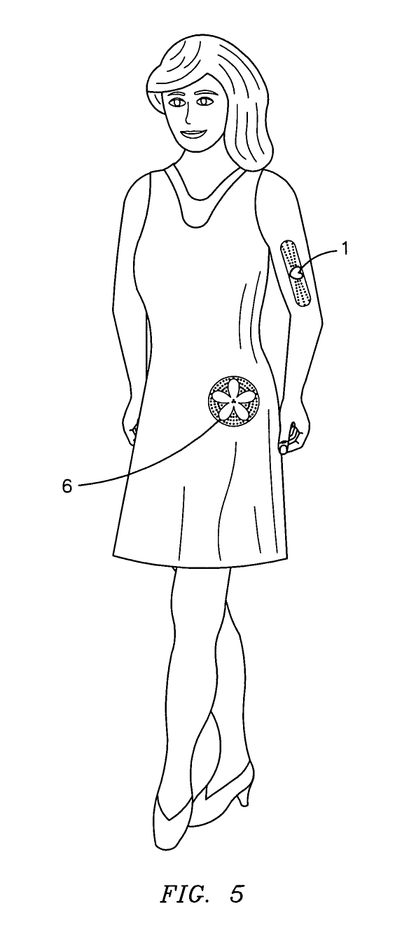

In FIGS. 3 and 4, various views of a decorative cover 6 of the present invention are shown. Similar to the decorative bandage 1, the decorative cover 6 of the present invention includes a top surface 2 and a bottom surface 3. The top surface 2 includes various decorative objects located thereon, such as crystals 4, glitter 8 and a symbol 7 in the shape of a flower. Similar to the decorative bandage 1, the bottom surface 3 of the decorative cover 6 is adhesive so as to stick to another surface.

Finally, FIG. 5 shows a perspective view of the decorative bandage 1 and decorative cover 6 of the present invention being worn on a person. As shown, a person may wear the decorative bandage 6 over an injury or blemish so as to conceal and protect the injury or blemish. Because the decorative bandage 6 includes a plurality of decorative elements located on the top surface 3 thereof, rather than having the appearance of a flat bandage, the decorative bandage 1 appears to be artwork that is directly applied onto a person's skin. In this manner, other individuals focus on the artwork and think that the person deliberately applied crystals 4 or other decor to the skin, similar to crystal tattoos and temporary body jewelry, rather than focusing on the bandage.

In addition, the decorative covers 6 may be applied to a person's clothing so as to mask stains or spots on the clothing. In this manner, individuals believe that the decorative covers 6 are part of the clothing itself, rather than a stain cover-up.

The use of the present invention will allow a person to mask an injury or blemish tastefully.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

Having thus described my invention, I claim:

1. A decorative bandage and cover comprising:
a substantially planar top surface and a bottom surface;
said bottom surface being self-adhesive; and
at least one decorative element located on said top surface.

2. The decorative bandage and cover of claim 1 further comprising:
a cushion located on said bottom surface.

3. The decorative bandage and cover of claim 1 wherein:
said at least one decorative element is a crystal.

4. The decorative bandage and cover of claim 1 wherein:
said at least one decorative element is a symbol.

5. The decorative bandage and cover of claim 1 wherein:
at least one decorative element is glitter.

6. The decorative bandage and cover of claim 2 wherein:
said at least one decorative element is a crystal.

7. The decorative bandage and cover of claim 2 wherein:
said at least one decorative element is a symbol.

8. The decorative bandage and cover of claim 2 wherein:
at least one decorative element is glitter.

9. The decorative bandage and cover of claim 4 wherein:
said at least one three-dimensional element is a crystal.

10. The decorative bandage and cover of claim 5 wherein:
said at least one decorative element is a crystal.

11. The decorative bandage and cover of claim 7 wherein:
said at least one decorative element is a crystal.

12. The decorative bandage and cover of claim 8 wherein:
said at least one decorative element is a crystal.

13. The decorative bandage and cover of claim 5 wherein:
said at least one decorative element is a symbol.

14. The decorative bandage and cover of claim 8 wherein:
said at least one decorative element is a symbol.

15. A decorative bandage and cover comprising:
a substantially planar top surface and a bottom surface;
said bottom surface being self-adhesive; and
at least one crystal located on said top surface.

16. A decorative bandage and cover comprising:
a substantially planar top surface and a bottom surface;
said bottom surface being self-adhesive;
at least one crystal located on said top surface; and
a cushion located on said bottom surface.
